(19) United States
(12) Patent Application Publication
(10) Pub. No.: US 2007/0163582 A1
(43) Pub. Date: Jul. 19, 2007

(54) Hand-held capsule device

(75) Inventor: Paul Kenneth Rand, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Middlesex (GB)

(21) Appl. No.: 10/595,615

(22) PCT Filed: Nov. 1, 2004
(86) PCT No.: PCT/EP04/12356

§ 371 Date: May 1, 2006

(30) Foreign Application Priority Data
Nov. 3, 2003 (GB) 0325628.6

(51) Int. Cl. A61M 15/00; A61M 15/08

(52) U.S. Cl. 128/203.23; 128/203.15

(57) ABSTRACT

In one aspect, the present invention provides a track (17; 117) adapted for use in a hand-held, capsule-containing device (1; 101) which is adapted to receive a series of capsules (21; 121) therein and defines a conveying path along which the capsules are conveyable. The path includes at least one fold section (23; 123) thereby to provide the path with a space-saving configuration. In another aspect of the present invention there is provided a hand-held device incorporating the track and capsules in the track. The capsules may contain a medicament power.

### FIELD OF THE INVENTION

The present invention relates to a hand-held capsule device and is particularly, but not exclusively, concerned with such a device for use in a dry powder inhaler in which the capsules each contain an inhalable medicament powder.

### BACKGROUND OF THE INVENTION

Dry powder inhalation devices (“DPI” for short) are well established for use in treating respiratory diseases. As an example, there may be mentioned the DISKUS® device of GlaxoSmithKline. In general, the pharmaceutical composition is formulated as a respirable powder and the powder is divided into a plurality of unit doses, each dose contained in its own sealed enclosure, for example blisters on a dosing strip. In use of the inhaler, the enclosures are opened, one at a time, by an opening mechanism of the inhalation device and the powder dose entrained into a patient's respiratory tract by an airflow generated through the device by the patient inhaling at a mouthpiece of the device.

The present invention proposes novel concepts having potential application in a DPI.

### SUMMARY OF THE INVENTION

According to the present invention there is provided a track adapted for use in a hand-held, capsule-containing device which is adapted to receive a series of capsules therein and defines a conveying path along which the capsules are conveyable, the path including at least one fold section thereby to provide the path with a space-saving configuration.

The present invention further provides a hand-held device for conveying capsules therein having a track according to the invention.

Preferably, the hand-held device is adapted for use as a component of an inhalation device for delivering medicament to a patient.

Preferred features of the invention are set forth in the subordinate claims appended hereto, as well as in the non-limiting exemplary embodiments of the invention hereinafter described with reference to the accompanying FIGURES of drawings.

### BRIEF DESCRIPTION OF THE DRAWINGS

FIG. 1 illustrates a first hand-held device according to the present invention.

FIG. 2 is an exploded perspective view of the first hand-held device without a capsule chain for better understanding.

FIG. 3 is a plan view of the first hand-held device with the upper face removed to better show a capsule chain in the device.

FIG. 4 is a cross-sectional side view of the first hand-held device along line IV-IV in FIG. 3.

FIG. 5 is a schematic view illustrating a conveying mechanism for the capsule chain provided in the first hand-held device.

FIGS. 6A-6F are a sequence of plan views corresponding to FIG. 3 showing the capsule chain as it moves through a complete circuit in the first hand-held device.

FIG. 7 is a plan view of a second hand-held device according to the present invention with its upper face removed to better show a capsule chain in the device.

FIG. 8 is a cross-sectional side view of the second hand-held device along line VIII-VIII in FIG. 7.

FIG. 9 is a cross-sectional underneath view of the second hand-held device along line IX-IX in FIG. 8.

FIG. 10 is a side view of one of the capsules in the capsule chain in the second hand-held device.

FIG. 11 is an end view of the capsule of FIG. 10 on arrow X.

FIG. 12 is an end view of the capsule of FIG. 10 on arrow Y.

FIG. 13 is a longitudinal section through two linked capsules of the capsule chain of the second hand-held device.

FIGS. 14A-E are a sequence of plan views corresponding to FIG. 7 showing the capsule chain as it moves through a complete circuit in the second hand-held device.

### DETAILED DESCRIPTION OF THE DRAWINGS

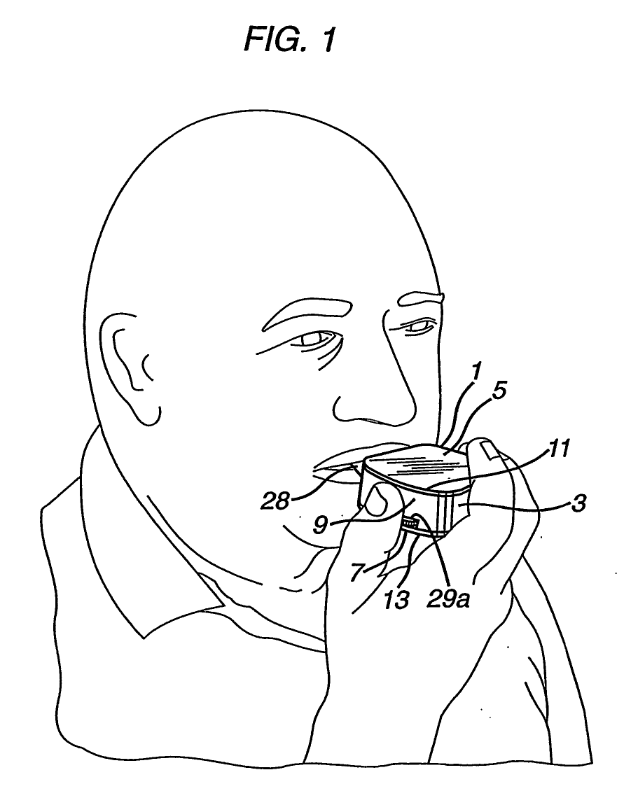

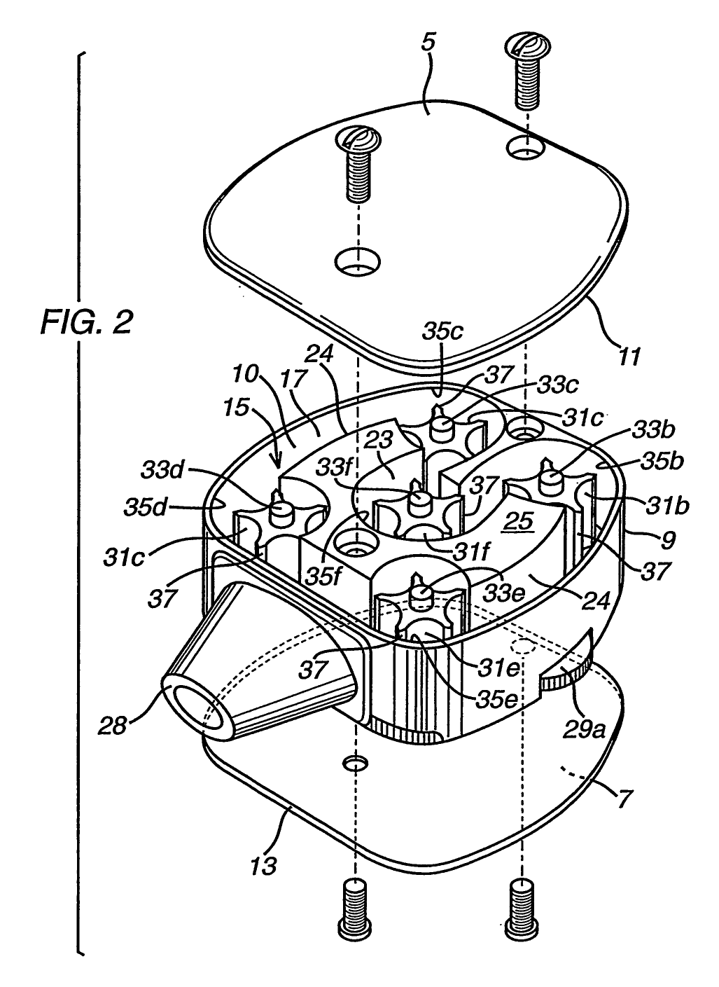

FIGS. 1-6 show a first hand-held device 1 in accordance with the present invention. The device 1 has a housing 3, in this embodiment made from a plastics material, optionally formed by moulding. The housing 3 has an upper face 5, a lower face 7 and an endless side face 9 which connects the outer peripheral edges 11,13 of the upper and lower faces 5,7, respectively. In this way, as shown in FIG. 2, the upper, lower and side faces 5,7,9 bound an inner volume 15 of the housing 3.

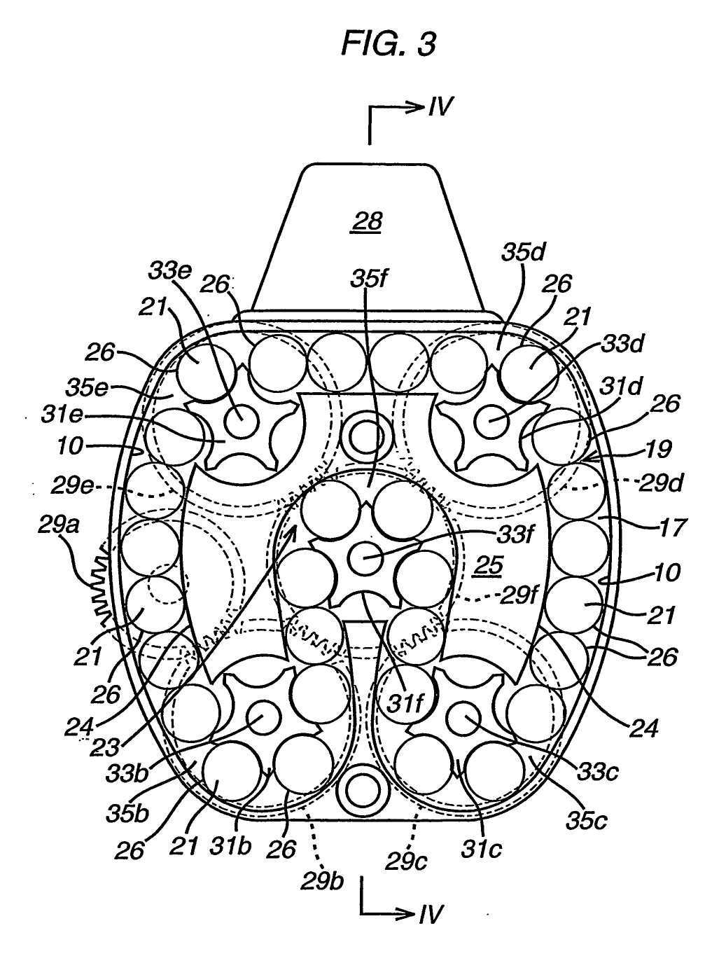

As shown in FIGS. 2 and 3, in the housing inner volume 15 there is provided an endless track 17 which receives an endless chain 19 of unlinked capsules 21. The track 17 has a path which is disposed adjacent the outer periphery of the housing 3 other than at a generally U-shaped fold section 23 of the track 17 which extends inwardly. The fold section 23 forms a loop or chicane in the track 17. The plan view of FIG. 3 shows that the fold section 23 gives the track a closed W-shape configuration.

The upper and lower faces 5,7 respectively present a roof 18 and a base 20 of the track 17. Moreover, the sides of the track 17 are presented by an inner surface 10 of the housing side face 9 and an opposing side face 24 of an inner wall structure 25 in the housing inner volume 15. The inner wall structure 25 may be of a plastics material, for instance made by moulding. Moreover, the inner wall structure 25 may be integrally formed with one of the other parts of the housing 3.

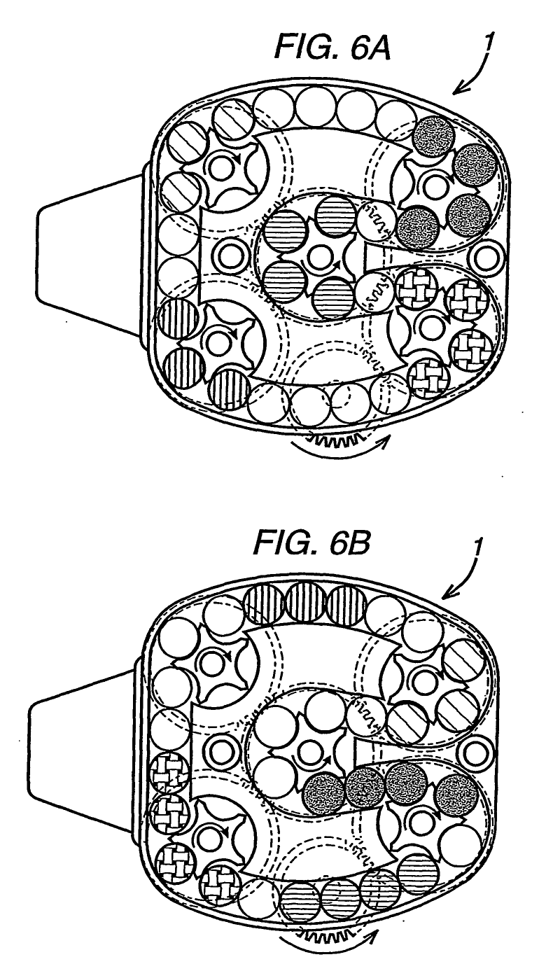

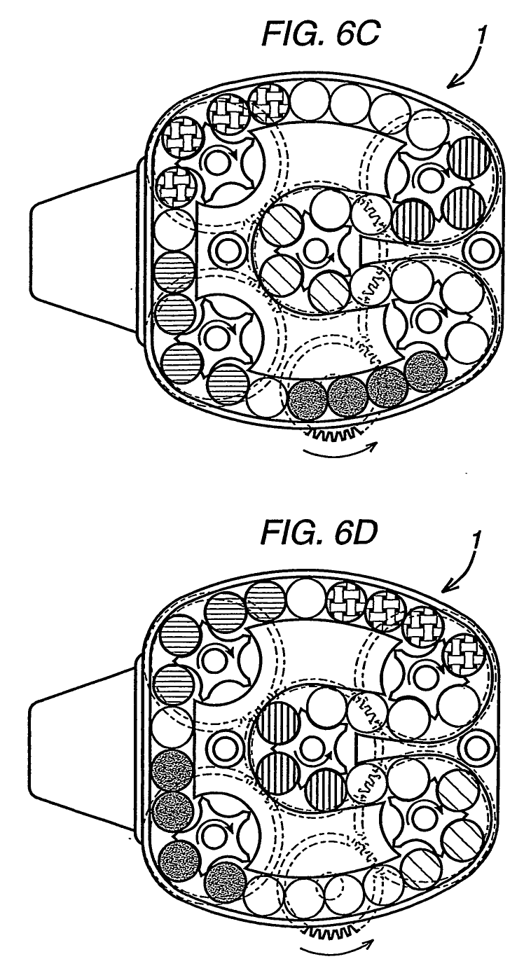

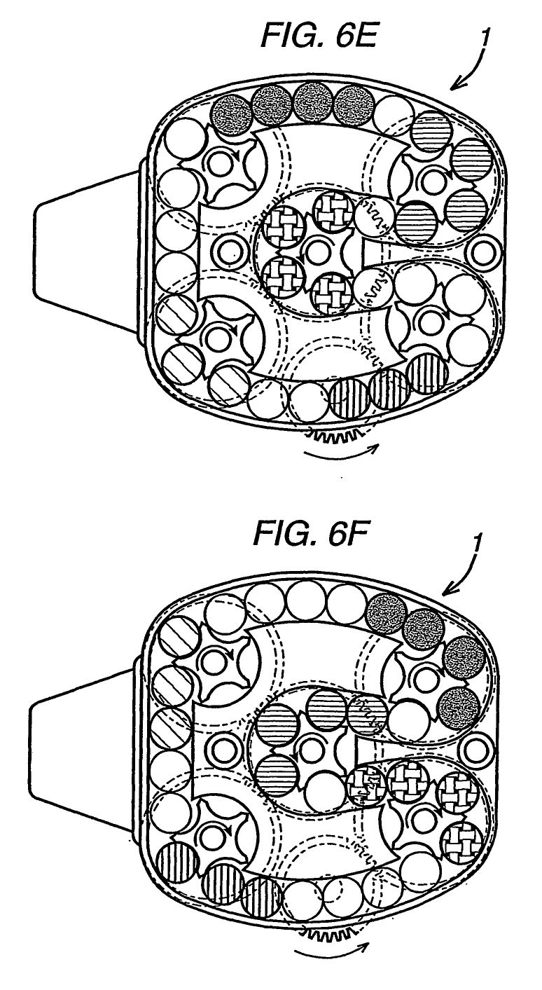

As will be seen from FIGS. 3 and 6, the capsules 21 are the same, with each comprising a hollow, generally cylindrical tube 26. In this embodiment the capsules 21 are made from a plastics material, preferably by moulding. The capsules 21 are disposed upright in the track 17 in side-by-side relation. The capsules 21 are adapted to receive a powder content therein, for example a medicament powder, and may take the form shown and described in WO2004/045688, the entire content of which is hereby incorporated herein by reference.

Where the capsules 21 each contain a dose of an inhalable medicament powder, the device 1 may take the form of a dry powder inhaler (DPI), as indicated by the provision of a mouthpiece 28 on the housing 3. The mouthpiece 28 could be replaced by another form of nozzle, for instance a nozzle sized and shaped for insertion into a nasal cavity.

Each capsule 21 may have a length (height) in the range of about 5 mm to about 15 mm and an outer diameter in the range of about 3 mm to about 8 mm. In other words, the capsules 21 may be referred to as a “microcapsule”. Such capsules 21 may be suited for holding a unit dose of a medicament powder in the range of about 2 μg to about 30 mg. The capsules 21 may contain a unit dose of pure active drug substance, or a blend of pure active drug substances, in the range of about 2 μg to about 250 μg (i.e. no bulk filler), or a bulked out unit dose of a medicament powder up to about 30 mg.

For a small unit dose of medicament powder, for instance in the range of about 2-250 μg, it is preferable for the capsules 21 to have a length (height) in the range of about 5 mm to about 6 mm and an outer diameter in the range of about 3 mm to about 5 mm.

Referring particularly to FIG. 5, the housing 3 is provided with a conveying mechanism for conveying the capsule chain 19 around the track 17. The conveying mechanism comprises a gear train 27 comprising six spur gear wheels 29a-f rotatably mounted in the housing 3. The gear wheels 29a-f in the embodiment are of a plastics material, optionally formed by moulding.

One of the gear wheels 29a (hereinafter the “actuator gear wheel”) protrudes from the housing side face 9 thereby enabling a user of the device 1 to cause rotation thereof with one of the fingers (e.g. thumb) of their hand holding the device 1 (see FIG. 1).

The other gear wheels 29b-f (hereinafter the “auxiliary gear wheels”) mesh with selected ones of the other auxiliary gear wheels and the actuator gear wheel 29a such that rotation of the actuator gear wheel 29a results in concurrent rotation of each of the auxiliary gear wheels 29b-f. Specifically, in this embodiment the central auxiliary gear wheel 29f meshes with each of the other auxiliary gear wheels 29b-e, which can be considered as satellite auxiliary gear wheels. Moreover, one of the satellite auxiliary gear wheels 29b meshes with the thumbwheel 29a. In this way, rotation of the thumbwheel 29a causes rotation of each auxiliary gear wheel 29b-f.

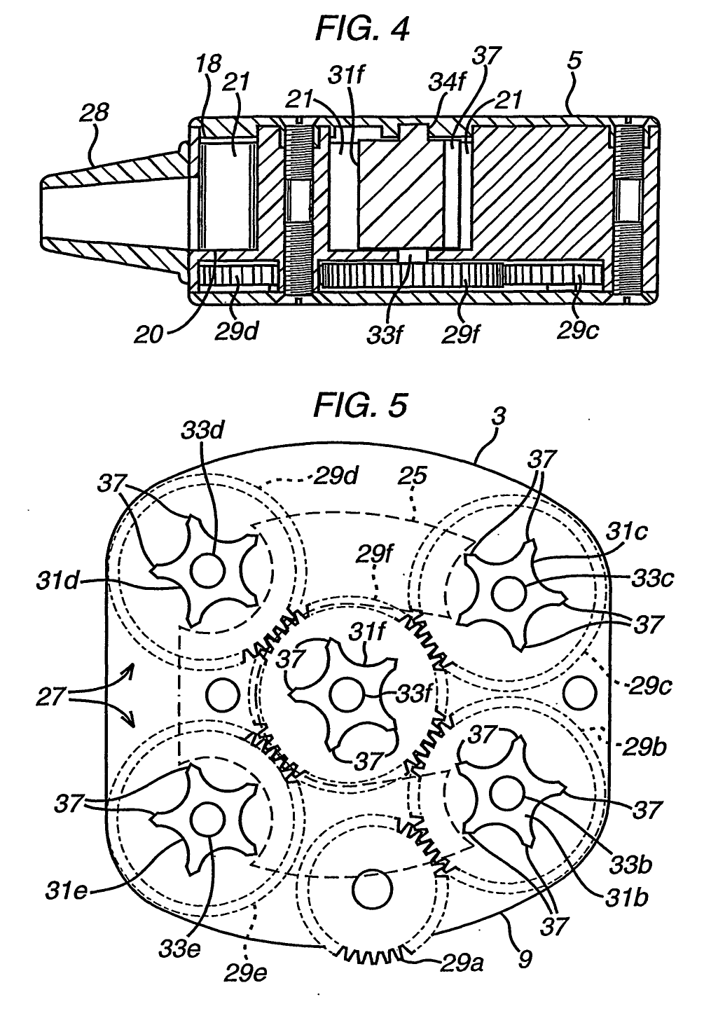

As will be further seen from FIGS. 4 and 5, each auxiliary gear wheel 29b-f is rotatably connected to a star wheel or a sprocket 31b-f. More particularly, each sprocket 31b-f has a spindle 33b-f which is mounted at one end thereof to the associated auxiliary gear wheel 29b-f at its axis of rotation. The other end of each spindle 33b-f is rotatably mounted in a recess in the roof 18 (the recess 34f for the centrally-located sprocket 33f is shown in FIG. 4). In this embodiment, the sprockets 31b-f are formed of a plastics material, optionally by moulding.

As will be appreciated, when the auxiliary gear wheels 29b-f are driven by the actuator gear wheel 29a, this results in rotation of the sprockets 31b-f. As will be appreciated, the sprockets 31b-f all rotate concurrently.

As will be understood from FIG. 2, each sprocket 31b-f is positioned at a bend 35b-f in the track 17 such that its teeth 37 engage the capsules 21 at the respective bend. Accordingly, when the sprockets 31b-f rotate, in response to the thumbwheel 29a being turned to cause rotation of the auxiliary gear wheels 29b-f, the sprocket teeth 37 advance the capsule chain 19 in the track 17.

FIGS. 6A-F show a full circuit of the capsule chain 19 in the track 17, with the capsules 21 in different segments of the capsule chain 19 being coded differently in FIGS. 6A-F to better illustrate the capsule movement. As shown by the arrows in FIGS. 6A-F, the rotation of the thumbwheel 29a in one rotative sense causes the capsule chain 19 to be driven by the conveying mechanism through the track 17 in the opposite rotative sense.

It will be appreciated that the provision of the fold section 23 in the track 17 provides the track with an increased path length compared to the case where the track 17 simply follows the outer periphery of the housing 3. Expressed another way, the fold section 23 gives the track 17 a compact, space-saving configuration. Accordingly, the track 17 is able to receive more capsules 21. When the device 1 is a dry powder inhaler, for instance, this means that the device is able to carry more doses of the powder medicament meaning that it will not need to be replaced by a patient so frequently.

It will also be appreciated by the skilled reader in the art that each gear wheel 29a-f in the gear train 27 could be replaced by a smooth-surfaced wheel with drive being transmitted along the train, and hence to the sprockets 31b-f, by frictional engagement between the wheels, i.e. through rolling contact between the wheels at respective pitch points.

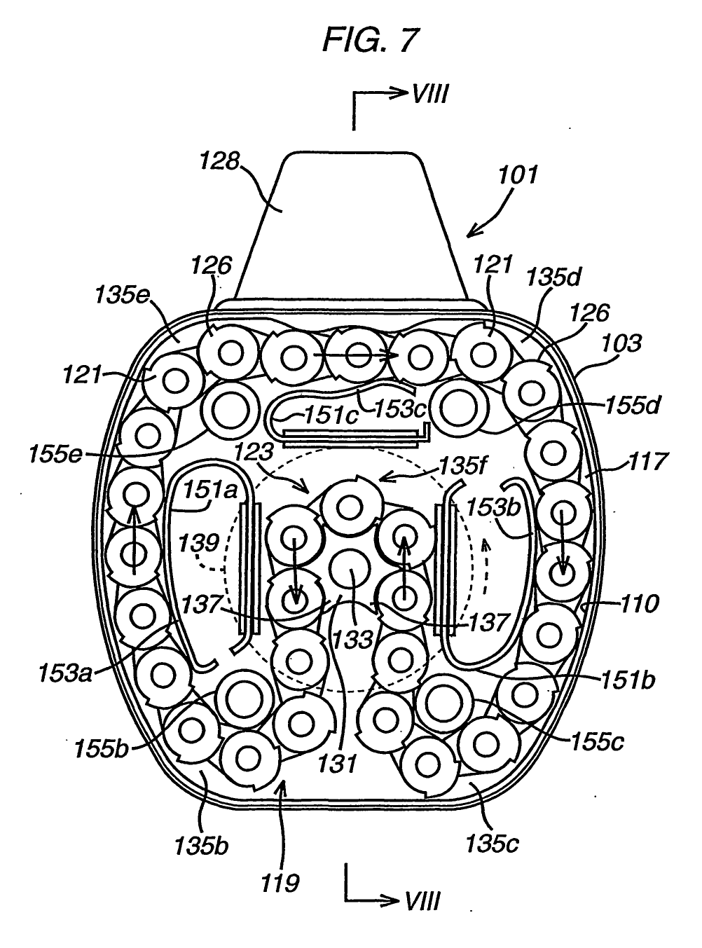

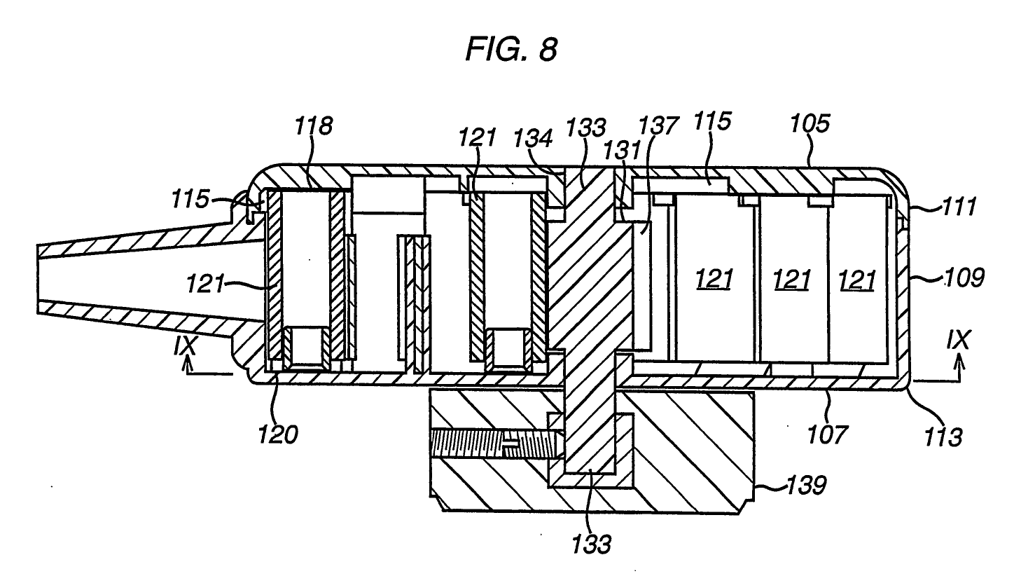

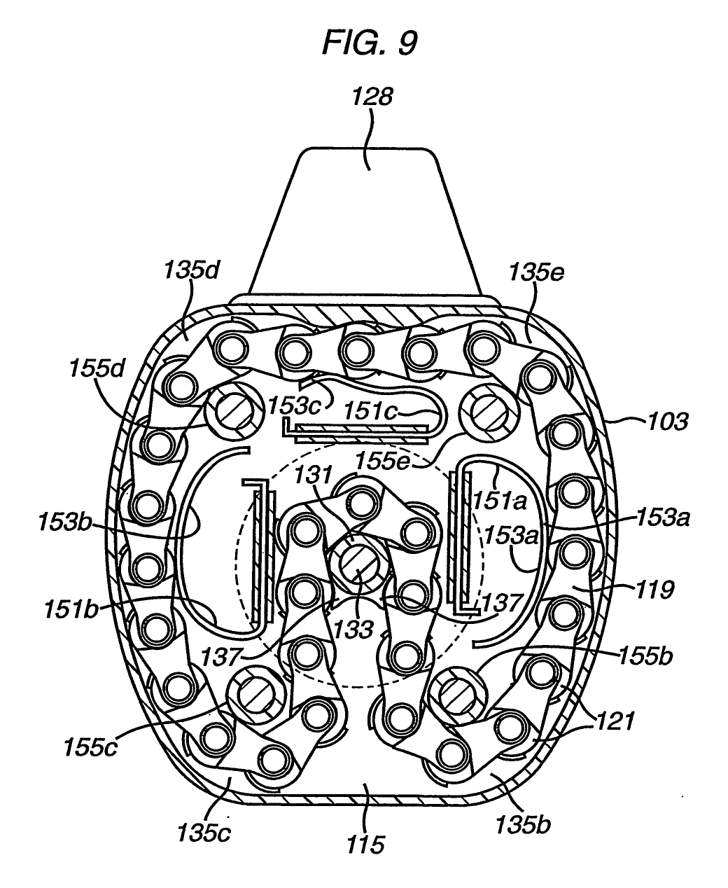

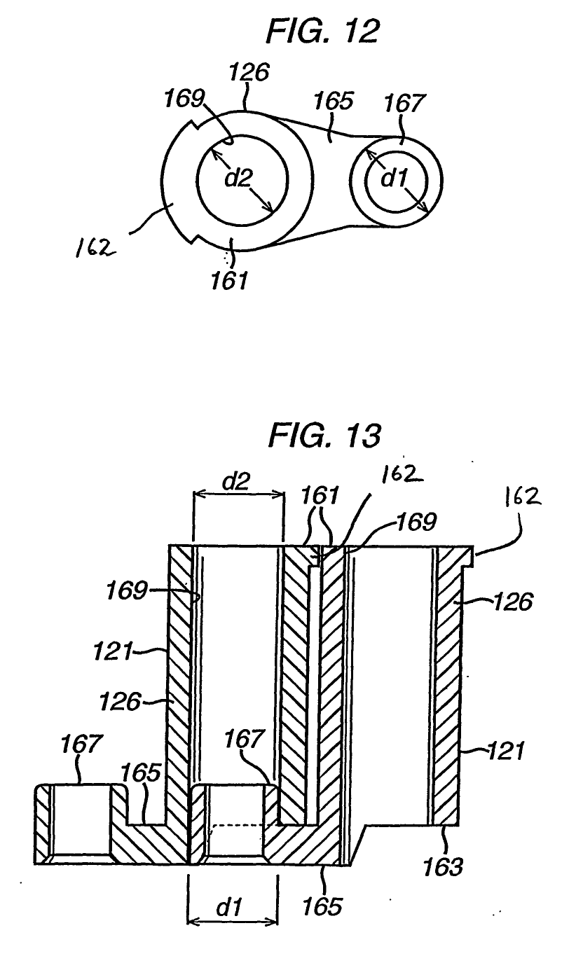

In FIGS. 7-14 there is shown a second hand-held device 101 in accordance with the present invention. The second hand-held device 101 corresponds closely to the first hand-held device 1. Accordingly, like features are identified by like reference numerals and no detailed description of the common features in the second device 101 will be given.

In the second device 101 the track 117 has a capsule chain 119 which is constituted by chain-linked capsules 121. That is to say, the capsules 121 in the chain 119 are linked together, not detached as in the first device 1. More particularly, the capsules 121 are linked into the chain 119 such that the chain 119 can be bent to go round the bends 135b-f of the track 117.

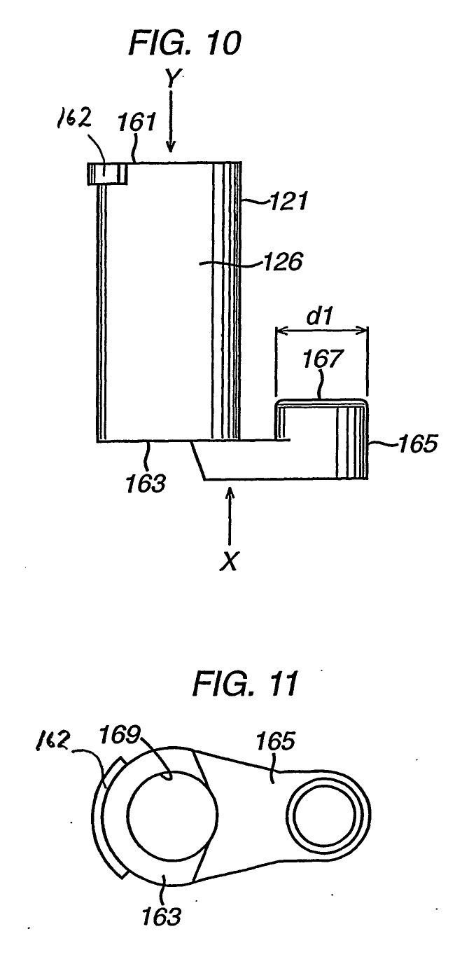

FIGS. 10-12 show one of the capsules 121 in the capsule chain 119 in greater detail. The hollow cylindrical tube 126 has an upper end 161 and a lower end 163 which is spaced longitudinally from the upper end 161. The tube 126 is provided with a foot 165 which extends radially outwardly from the lower end 163 and has an upstanding circular boss 167.

As shown in FIG. 13, the foot 165 provides the linkage for the capsule chain 119 inasmuch as the boss 167 of each capsule 121 is pluggable into the lower end of the lumen 169 of an adjacent capsule 121 in the chain 119 so as to link the capsules 121 together. Moreover, the relative dimensioning of the boss 167 and the lumen 169 enables the capsules 121 to pivot about the boss 167 inserted thereinto thereby enabling the capsule chain 119 to negotiate the bends 135b-f in the track 117.

Preferably, the boss 167 has an outer diameter d1 which is equal to, or marginally less than, the inner diameter d2 of the circular lumen 169 of the tube 126.

At the upper end 161 of the cylindrical tube 126 there is provided a radial lip segment 162. As will be appreciated from FIGS. 7 and 13, the purpose of the lip segments 162 is to prevent, or substantially prevent, the capsules 121 tilting about their longitudinal axes when linked into the capsule chain 119 by bearing against the neighbouring capsules 121 in the chain 119.

Further information on the capsules 121, and on different forms they may take, is contained in Applicant's co-pending International patent application No. PCT/EP2004/004007 filed on 14 Apr. 2004 claiming priority from UK patent application No. 03 089 69.5 filed on 17 Apr. 2003, the entire contents of each of which are hereby incorporated herein by reference.

The capsules 121 in the second device 101 may be of corresponding dimensions to those mentioned previously for the capsules 21 of the first device 1. Moreover, the lumen 169 of each capsule 121 may have an inner diameter d2 in the range of about 1 mm to about 6 mm. For a small unit dose of pharmaceutical powder, for instance in the range of about 2-250 μg, it is preferable for the lumen inner diameter d2 to be in the range of about 1 mm to about 3 mm, more preferably about 2 mm.

As shown in FIG. 7, for example, the inner surface of the track 117 in the second device 101 is not defined by a central insert, as in the first device 1. Instead, the second device 101 has a plurality of generally U-shaped clips 151a-c clipped thereinto. The resilient outer limb 153a-c of each clip 151a-c defines the side sections of the track 117. Moreover, on the inside of each of the track bends 135b-e is disposed a pillar 155b-e about which the capsule chain 119 is wound.

Having the capsules 121 linked together into the chain 119 enables the conveying mechanism of the device 101 to be simplified compared to that used in the first device 1. In this embodiment, the conveying mechanism comprises a single sprocket 131 for advancing the capsule chain 119. For convenience, the sprocket 131 is located on the inside of the bend 135f of the fold section 123 of the track 117. The spindle 133 of the sprocket 131 is rotatably connected to a knob 139, preferably having a knurled outer surface, disposed under the lower face 107 of the housing 103. Thus, rotation of the knob 139 causes rotation of the sprocket 131 and advancement of the capsule chain 119 in the track 117.

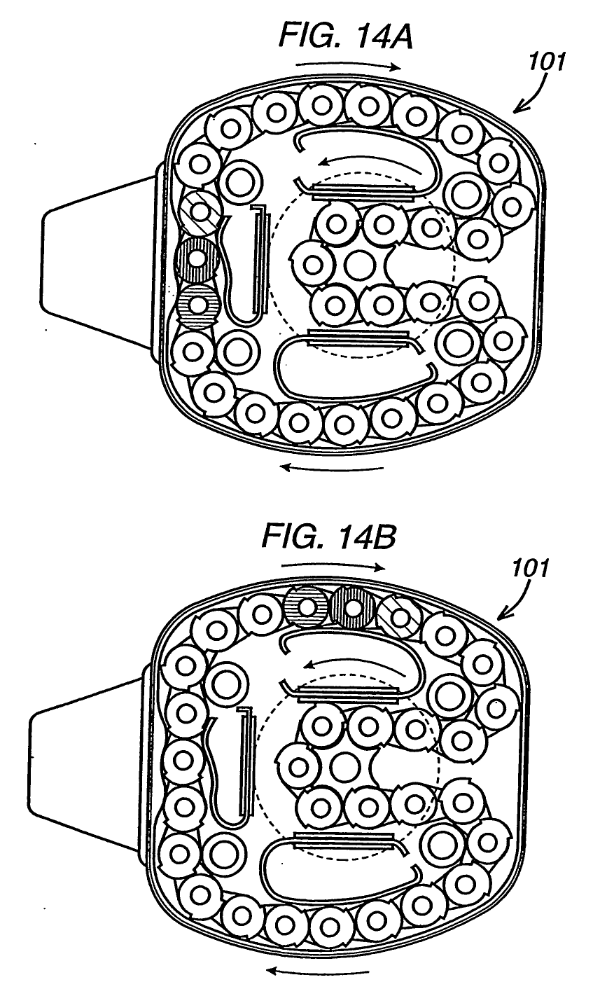

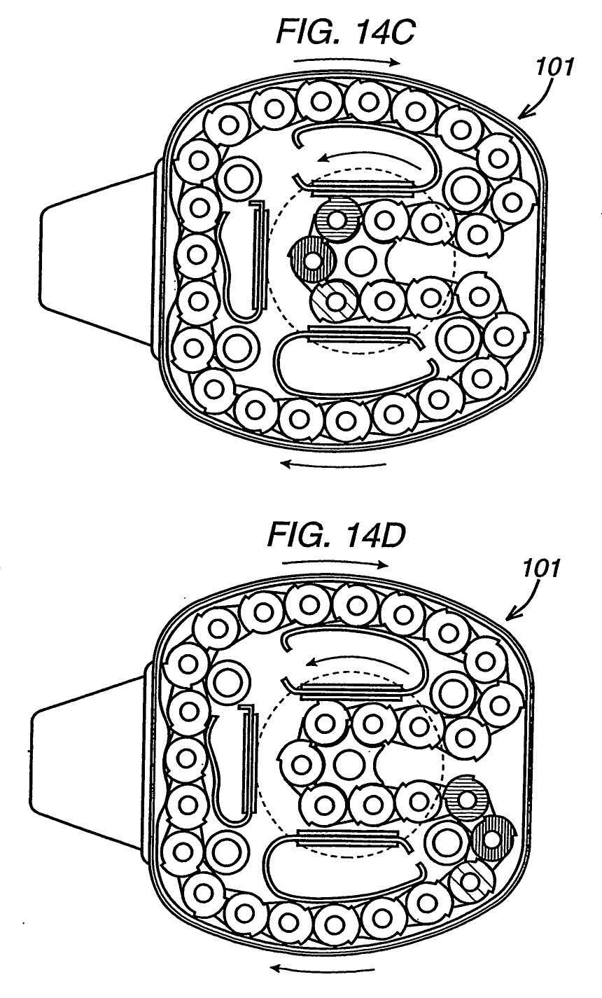

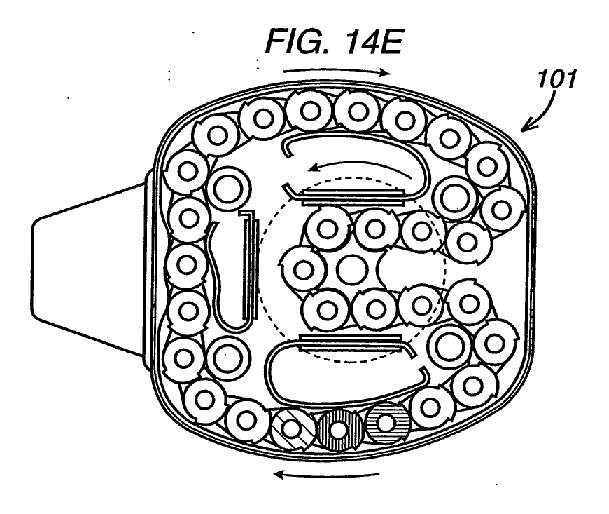

FIGS. 14A-E show the sequence of movement of the capsule chain 119 through a complete circuit of the track 117 in response to rotation of the knob 139. As indicated by the arrows, the capsule chain 119 circulates the track 117 in an opposite rotative sense compared to the knob 139.

Appropriate medicaments for the medicament powder for use in the present invention may be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]-ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}-pentanoyl)amino] propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred medicaments are an anti-inflammatory agent (for example a corticosteroid or an NSAID), an anticholinergic agent, a β₂-adrenoreceptor agonists, an antiinfective agent (e.g. an antibiotic or an antiviral) and an antihistamine. The medicament may be the sole medicament in the capsules or in combination with another medicament. Preferred combinations are based on the preferred medicament list above.

Preferred as a component of a medicament combination in the capsules are albuterol, salmeterol, fluticasone propionate and beclomethasone dipropionate and salts or solvates thereof, e.g., the sulphate of albuterol and the xinafoate of salmeterol.

A particularly preferred medicament combination for use in the capsules of the invention is a bronchodilator in combination with an anti-inflammatory. The bronchodilator is suitably a beta-agonist, particularly a long-acting beta-agonist (LABA). Suitable bronchodilators include salbutamol (e.g., as the free base or the sulphate salt), salmeterol (e.g., as the xinafoate salt) and formoterol (e.g. as the fumarate salt). The anti-inflammatory is suitably an anti-inflammatory steroid. Suitable anti-inflammatory compounds include a beclomethasone ester (e.g., the dipropionate), a fluticasone ester (e.g., the propionate) or budesonide or any salt or solvate thereof. One preferred combination is fluticasone propionate and salmeterol, or any salt or solvate thereof (particularly the xinafoate salt). A further preferred combination is budesonide and formoterol or any salt or solvate thereof (e.g. formoterol as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as a pure drug or together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient. The excipient may be included with the medicament via well-known methods, such as by admixing, co-precipitating and the like.

Particles of the powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

For the avoidance of doubt, the present invention is not limited to the specific embodiments described above with reference to the FIGURES of drawings, but may take any form within the scope of the appended claims. Moreover, the specific embodiments may be modified in accordance with the claims. Furthermore, the use of prefixes such as “generally” and the like in relation to parameters and features of the invention is meant to encompass the exact parameter or feature, as well as deviations therefrom. Lastly, the inclusion of reference numerals in the claims is solely for illustration, and not to be taken as having a limiting effect on the claims.

The present application claims priority from UK patent application No. 03 256 28.6 filed on 3 Nov. 2003, the entire original content of which is hereby incorporated herein by reference. The application is also related to the Applicant's concurrently filed International patent application entitled ‘A Hand-Held Capsule Device’ which claims priority from UK patent application No. 03 256 27.8 filed on 3 Nov. 2003, the entire contents of each of which are hereby incorporated herein by reference.

What is claimed is:

1. A track adapted for use in a hand-held, capsule-containing device which is adapted to receive a series of capsules therein and defines a conveying path along which the capsules are conveyable, wherein the path includes at least one fold section thereby to provide the path with a space-saving configuration.

2. A track according to claim 1, wherein the path is an endless path.

3. A track according to claim 1, wherein the at least one fold section is inwardly directed.

4. A track according to claim 2, wherein the at least one fold section is directed towards another section of the path.

5. A track according to any one of claim 1, wherein the at least one fold section is a loop section in the path.

6. A track according to claim 5, wherein the loop section is an inverted, generally U-shape section.

7. A track according to claim 1, wherein the path has a pair of opposed first side sections extending from first ends thereof generally in a first direction and a second side section extending generally transversely to the first direction to connect the respective first ends of the first side sections and wherein the at least one fold section is in the second side section and has a pair of opposed first side portions which extend generally in the first direction, the at least one fold section being directed such that each first side portion is in facing relation with a different first side section.

8. A track according to claim 7, wherein the first and second side sections define a generally W-shape when viewed in plan.

9. A track according to claim 1 adapted to receive the capsules in a chain configuration.

10. A track according to claim 1 adapted to receive, and to enable conveying of, elongate capsules oriented upright in the track.

11. A hand-held device for conveying capsules therein having a track according to claim 1.

12. A hand-held device according to claim 11 further having a plurality of capsules in the track.

13. A hand-held device according to claim 12 in which the capsules are in a chain configuration in the track.

14. A hand-held device according to claim 13 in which the capsules are in an endless chain in the track.

15. A hand-held device according to claim 13 in which the capsules are linked together to form the chain.

16. A hand-held device according to claim 12 in which the capsules are of elongate form and disposed upright in the track.

17. A hand-held device according to claim 12 in which the capsules are of generally cylindrical form.

18. A hand-held device according to claim 12 in which the capsules contain a powder.

19. A hand-held device according to claim 12, wherein the capsules contain a medicament.

20. A hand-held device according to claim 11 further having a conveying mechanism for conveying the capsules about the track.

21. A hand-held device according to claim 20 in which the conveying mechanism comprises at least one sprocket for driving the capsules about the track.

22. A hand-held device according to claim 21, wherein the at least one sprocket is located at a bend in the track for engaging the capsules for advancement thereof along the track.

23. A hand-held device according to claim 22, wherein the sprocket is located at the fold section.

24. A hand-held device according to claim 11 adapted for use as a component part of an inhalation device for delivering medicament to a patient.

25. An inhalation device for delivering medicament to a patient incorporating a hand-held device according to claim 11.

26.-27. (canceled)
